(19) United States
(12) Patent Application Publication
(10) Pub. No.: US 2013/0035357 A1
(43) Pub. Date: Feb. 7, 2013

(54) Compositions and Methods for Preventing Joint Destruction in Osteoarthritis

(71) Applicants: Richard Chou, West Lebanon, NH (US); Constance E. Brinckerhoff, New London, NH (US); Daniel A. Albert, Etna, NH (US)

(72) Inventors: Richard Chou, West Lebanon, NH (US); Constance E. Brinckerhoff, New London, NH (US); Daniel A. Albert, Etna, NH (US)

(73) Assignee: TRUSTEES OF DARTMOUTH COLLEGE, Hanover, NH (US)

(21) Appl. No.: 13/641,173

(22) PCT Filed: Apr. 13, 2011
(86) PCT No.: PCT/US11/32237

§ 371 Date: Oct. 15, 2012

Related U.S. Application Data
(60) Provisional application No. 61/324,350, filed on Apr. 15, 2010.

(51) Int. Cl. A61K 31/192; A61P 29/00; A61K 31/4706; A61P 19/02

(52) U.S. Cl. 514/313; 514/569

(57) ABSTRACT

The present invention is methods for inhibiting collagen destruction in joints of osteoarthritis patients. The methods are based on use of rexinoid compounds, in particular bexarotene, and their activity to inhibit synthesis of matrix metalloproteinases (MMPs) in affected tissue.

### BACKGROUND OF THE INVENTION

Osteoarthritis (OA) is the most common chronic disease of the joints, with no effective therapeutic treatments. It is considered a heterogeneous group of degenerative joint diseases associated with aging or mechanical stress, as seen in obesity. No genetic linkage has been generally established in OA (Firestein, G. 1997. In: W. Kelley et al. (Eds) Textbook of Rheumatology, W.B. Saunders:Philadelphia, pp. 851-897), although linkages between HLA haplotypes and subgroups of OA have been reported (Martel-Pelletier, J. et al. 2001. Best Pract. Res. Clin. Rheumatol. 15:805-829; Mengshol, J. A. et al. 2002. Arth. Rheum. 46:13-20; Burrage, P. S. et al. 2007. Front. Biosci. 11:529-543).

Recent studies have demonstrated the inflammatory nature of OA, as evidenced by the presence of low-grade inflammation with leukocytic infiltrate and pro-inflammatory cytokines in the OA joints (Burrage, P. S. et al. 2007. Front. Biosci. 11:529-543). These changes result in chronic joint damage of cartilage and subchondral bones. Critical to this destruction is the pro-inflammatory cytokine Interleukin-1β (IL-1β) produced by cells within the diseased joint. IL-1≈ acts on connective tissue cells, resulting in an increased expression of matrix metalloproteinases (MMPs), a family of enzymes that degrades the extracellular matrix. Specifically, increases in collagenase-1 (MMP-1) and collagenase-3 (MMP-13) mediate joint destruction by degrading collagens, types I, II and III. Most connective tissue cells, including synovial fibroblasts and chondrocytes, express MMP-1, while MMP-13 expression is usually restricted to bone cells and chondrocytes. Since joint destruction outpaces repair of cartilage and joint tissues in OA, therapeutic strategies to block these enzymes are attractive. Currently, no such therapy is available.

Significant effort has been invested in attempts to design effective inhibitors of MMP activity and/or synthesis, with the idea of tempering or even eliminating pathologic levels of enzyme activity could provide significant clinical benefit. However, the highly conserved structure of the MMP catalytic domain has made it difficult to maintain target specificity, and many compounds display a promiscuous inhibitor profile, with efficacy against multiple MMP family members. The lack of selectivity has been implicated in the significant side effects seen in clinical trials of MMP inhibitors. Thus, inhibition of MMP synthesis has been an alternative focus. Compounds that target MMP synthesis have been identified, including the retinoids, a class of vitamin A derivatives (Sporn, M. B. and A. B. Roberts. 1985. Ciba Found. Symp. 113:1-5). Retinoids are ligands for a family of nuclear hormone receptors and are thought to inhibit MMP production by affecting the levels of AP-1 proteins (Schroen, D. J. and C. E. Brinckerhoff. 1996. Gene Expr. 6:197-207). Although retinoids are effective inhibitors of MMP synthesis, they have pleiotropic effects that lead to significant clinical toxicities (Nesher, G. and J. Zuckner. 1995. Semin. Arth. Rheum. 24:291-296). Efforts to minimize toxicities while maintaining the desired regulatory effects have focused on molecules that selectively bind to the retinoid X receptors (RXRs). These compounds, called rexinoids, elicit regulatory effects while escaping some of the toxicities (Hede, K. 2004. J. Natl. Cancer Inst. 96:1807-1808). Recently, one rexinoid, LG100268 (LG268) was shown in in vitro experiments to inhibit proinflammatory cytokine-induced upregulation of MMP-1 and MMP-13 by decreasing the transcription rates of these genes, as well as to blunt the ability of IL-1β-stimulated SW-1353 cells to degrade collagen matrix (Burrage, P. S. et al. 2007. Arth. Rheum. 56:892-904). Of particular interest was the fact that the rexinoid compound targeted inhibition of MMPs that was linked to pro-inflammatory cytokine-driven expression (e.g., MMP-1 and MMP-13) rather than on expression of constitutively expressed MMPs (e.g., MMP-2 and MMP-14). As a result, the data indicated that rexinoid compounds may have utility as therapeutics to inhibit MMP synthesis in vivo.

Recent data suggests that expression of certain nuclear receptors, wherein RXR is such a nuclear receptor, is decreased in osteoarthritis-affected cartilage (Collins-Racie, L. A. et al. 2009. Osteoarth. Cart. 17:832-842). The authors examined transcriptional levels of 48 different nuclear receptors in samples of human articular cartilage from patients with OA as well as from controls (no OA). They reported dysregulation of certain nuclear receptor expression patterns, with liver X receptor (LXR) signaling showing significant impairment in tissue from OA patients. Treatment with LXR-modulating compounds is suggested as a novel treatment strategy for OA.

### SUMMARY OF THE INVENTION

The present invention is a method of inhibiting synthesis of matrix metalloproteinase (MMP) in synovial tissues comprising contacting synovial tissue with an effective amount of a rexinoid drug so that levels of MMP in synovial tissue are decreased. In a preferred embodiment the rexinoid drug is bexarotene.

A method for inhibiting the degradation of collagen matrix in synovial tissue comprising contacting synovial tissue with an effective amount of a rexinoid drug so that the degradation of collagen is inhibited. In a preferred embodiment the rexinoid drug is bexarotene.

A method for treating the symptoms of osteoarthritis in a patient comprising administering to a patient suffering from symptoms of osteoarthritis an effective amount of a rexinoid drug so that the symptoms of osteoarthritis are reduced. In a preferred embodiment the rexinoid drug is bexarotene. In one embodiment the symptoms of osteoarthritis to be treated are the degradation of collagen in synovial tissue of the joints of the patient. In yet another embodiment the drug is administered orally, or in combination with a non-steroidal anti-inflammatory drug or hydroxychloroquine.

### DETAILED DESCRIPTION OF THE INVENTION

The present invention is a method for treating the symptoms of osteoarthritis and joint destruction in osteoarthritis due to collagen degradation in the joints. The method involves administration of a rexinoid drug, such as bexarotene, to patients, wherein the rexinoid drug has been shown to inhibit the degradation of collagen in synovial tissue through its activity to inhibit synthesis of matrix metalloproteinase (MMP). In the context of the present invention a “rexinoid” drug is any drug shown to have pharmacologic activity to inhibit retinoid X receptors in cells or tissues. Such compounds include but are not limited to the synthetic rexinoids bexarotene (also known as LGD1069 and TARGRETIN®) and LG268, as well as naturally-occurring compounds such as phytanic acid.

Bexarotene (TARGRETIN®) has been approved by the U.S. Food and Drug Administration (FDA) for the oral treatment of cutaneous T-cell lymphoma. It is currently undergoing clinical testing for other forms of cancer (Qu, L. and X. Tang. 2010. Cancer Chemother. Pharmacol. 65:201-205). Bexarotene is a selective RXR agonist which produces its pharmacological effects in cancer patients presumably through blocking cell cycle progression, inducing apoptosis and cell differentiation, preventing multi-drug resistance, and inhibiting angiogenesis and metastasis. It acts as an agonist compound at all three types of retinoid X receptors, RXRα, RXRβ, and RXRγ. Unlike some other retinoid compounds, the rexinoid bexarotene has been shown to have negligible activity to bind retinoid acid receptors (RARs; e.g., RARα, RARβ, and RARγ) and to transactivate the related RAR genes. U.S. Pat. No. 5,780,676, U.S. Pat. No. 5,962,731, and U.S. Pat. No. 6,043,279 disclose the chemical structure of bexarotene and some of its derivatives as well as their use to treat diseases such as cancer, psoriasis, acne, and autoimmune disease, such as rheumatoid arthritis, as well as the activity of these compounds to modulate cholesterol levels and lipid metabolism in blood. In all cases, the activity of bexarotene is linked to its selective activity to activate RXR receptors. Recent research has focused on the development of novel bexarotene analogues with improved safety profiles (Wagner, C. E. et al. 2009. J. Med. Chem. 52:5950-5966). Such bexarotene analogues include compounds such as trifluoromethyl bexarotene, a thiocarbamte bexarotene analogue, cyclopropyl dienoic acid, as well as novel aza retinoids and amide retinoids, the structures of which are listed by Wagner et al. (2009. J. Med. Chem. 52:5950-5966).

It has now been found that rexinoids, including bexarotene, have activity apart from their previously identified activity. The novel pharmacological activity of rexinoids relates to the ability to inhibit synthesis of MMP in synovial tissues, which leads to an inhibition of the degradation of collagen in the cartilage, lessening joint destruction. Experiments can be performed to elucidate these pharmacological activities and demonstrate the in vivo efficacy of the rexinoid bexarotene as a treatment for osteoarthritis as well as its safety in patients suffering from osteoarthritis.

In order to elucidate the pharmacological effects of bexarotene, experiments can be performed in vitro in human chondrocytic cells and synovial fibroblasts. It should be noted that human chrondrocytic cells are the model of choice for extrapolation to human in vivo efficacy since it had previously been shown that data collected in animal chondrocytes had poor clinical relevance (Kirchmeyer et al. 2008. Biorheology 45:415-432). The basic pharmacological profile of bexarotene is known in the art, in terms of its receptor binding affinity for RXR receptors (Wagner, C. E. et al. 2009. J. Med. Chem. 52:5950-5966). However, the pharmacological activity of bexarotene in chondrocytes or synovial fibroblasts in particular has not been established. In previous experiments, it was shown in a model system of human chondrocytic cells that a ligand for nuclear hormone receptors RXR significantly reduced MMP gene expression induced by inflammatory cytokines, IL-1β and tumor necrosis factor-α (TNF-α) (Burrage, P. S. et al. 2007. Arth. Rheum. 56:892-904). The RXR-specific ligand, LG268, is related to bexarotene (TARGRETIN®) (Liby, K. T. et al. 2007. Nat. Rev. Cancer 7:357-369; Rigas, J. R. and K. H. Dragnev. 2005. Oncologist 10:22-33; Smit, J. V. et al. 2004. J. Am. Acad. Derm. 51:257-264). The activity of LG268 as a selective RXR agonist has been demonstrated and compared with the potency of bexarotene (Boehm, M. F. et al. 1995. J. Med. Chem. 38:3146-3155). LG268 showed equal potency as an agonist at RXRα, RXRβ, and RXRγ receptors, and was almost an order of magnitude more potent than bexarotene as an RXR agonist (Boehm, M. F. et al. 1995. J. Med. Chem. 38:3146-3155). However, the comparative potency of LG268 and bexarotene in the inhibition of collagen breakdown in vivo has not been reported.

LG268 was shown to inhibit production of mRNA and protein for MMP-1 and MMP-13, and prevented breakdown of collagen by these cells in vitro (Burrage, P. S. et al. 2007. Arth. Rheum. 56:892-904). Suppression of collagen breakdown was shown to involve changes in transcription and chromatin remodeling (Burrage, P. S. et al. 2007. Arth. Rheum. 56:892-904). In the present invention, bexarotene will be used both in vitro and in vivo to examine inhibition of MMP synthesis in synovial tissue and the inhibition of collagen breakdown in joints of patients suffering from OA.

Inherent heterogeneity among patients with OA can result in variation in the levels of basal and cytokine-induced MMP expression and in the degree of suppression by RXR ligands. Therefore to demonstrate efficacy, initial experiments involve 1) monitoring basal expression of MMP-1 and MMP-13 mRNA and protein, as well as inflammatory markers, such as cytokines and chemokines, with microarray analysis, in synoviocytes and chondrocytes from OA patients; 2) measuring induction of MMP-1 and MMP-13 in primary cultures of human chondrocytes and synovial fibroblasts in response to proinflammatory cytokines IL-1β and tumor necrosis factor-α (TNFα); and 3) documenting the ability of the retinoid RXR agonist bexarotene, with or without two common anti-inflammatory agents (ibuprofen and hydroxychloroquine, agents commonly used to treat other forms of inflammatory arthritis), to reduce expression of MMPs and inflammatory markers, and block collagen degradation in vitro.

For initial in vitro experiments, synovial tissues can be obtained from patients who undergo total joint arthroplasty for OA and control subjects that are available commercially, and then processed for in vitro studies on the inhibition of MMP production in synovial fibroblasts and chondrocytes by bexarotene. The primary objective of these in vitro studies is to demonstrate the efficacy of the rexinoid bexarotene in the inhibition of MMP production in synovial tissues of OA patients. The results collected in tissues are used to guide the initiation of a clinical trial on the targeted inhibition of MMP production via the RXR pathway as a therapeutic treatment for OA. This clinical trial is designed to determine the efficacy of bexarotene when used in combination with therapeutic agents routinely administered to patients with OA. The results of these studies also elucidate the inflammatory nature of OA and the heterogeneity of MMP production in a mixed population of OA patients.

About 150 patients who undergo total hip/knee arthroplasty for OA are enrolled in the clinical trial. The study involves patients who undergo total hip arthroplasty (THA) or total knee arthroplasty (TKA) for an established diagnosis of end stage joint disease of the hips or knees due to OA. These patients agree to allow the investigators to perform a chart review on their history and radiographic findings in order to confirm the diagnosis and calculate the Sharp score, respectively. All patients are asked to fill out a questionnaire in order to determine their functional status prior to the procedure. Resected synovial tissues are collected from all patients during surgical procedures and then processed for in vitro studies. Patients are excluded from the study if they have established diagnoses of chronic inflammatory arthritis (such as rheumatoid arthritis, lupus arthritis, gouty arthritis, etc), septic arthritis, or malignancy of the joints.

Samples of synovial tissue and cartilage obtained during surgical intervention, mainly total joint arthroplasty, are maintained in primary cell cultures. Using RT-PCR and western blot analysis, concentration-effect curves are produced to test the ability of bexarotene in inhibiting MMP mRNA and protein production in cells in culture, similar to the methods described previously (Burrage, P. S. et al. 2007. Arth. Rheum. 56:892-904). Functional efficacy of bexarotene is determined using a collagen destruction assay (Burrage, P. S. et al. 2007. Arth. Rheum. 56:892-904). Since some patients may display higher constitutive expression of MMP as a result of diseased tissues characteristic of chronic OA, basal/constitutive MMPs are monitored as well as the ability of bexarotene to antagonize this expression and inhibition of MMP induction by IL-1β. Cell cultures are terminated at selected times, e.g. 12 hours, 24 hours, or 48 hours. To assess potential global effects of bexarotene on these cells, limited micro-array analysis using PROFILER® kits for inflammatory cytokines are also performed.

Once the effects of bexarotene as a single agent have been documented, the studies are extended to include combination treatments with drugs commonly used to treat symptoms of OA such as NSAIDs and hydroxychloroquine.

In all in vitro studies, the following three parameters are established for all of the different drug treatments. First, graded concentrations of bexarotene are used to establish the dose-response effects of bexarotene on MMP production in synoviocytes and chondrocytes of OA patients. The lowest dose of bexarotene required to provide the optimal inhibition of MMP production is defined. In addition, a comparison of the doses required to produce 50% inhibition of MMP synthesis (IC₅₀) among different OA patients, in conjunction with their pre-operative functional status, allows for identification of possible molecular mechanisms involved in the regulation of MMP production via the RXR pathway. Second, optimal doses of bexarotene are defined in order to determine the kinetics of MMP inhibition by bexarotene in synovial fibroblasts and chondrocytes of human OA patients. Third, the synergistic effects of inhibition of MMP production are defined in the presence of other common anti-inflammatory agents, including but not limited to NSAIDs, such as ibuprofen, and hydroxychloroquine. Such in vitro combined treatment testing is particularly important since many treatments for OA involve use of more than one agent.

Once the efficacy of a drug, such as bexarotene, or a combination of drugs, has been shown based on the use of the in vitro screening, clinical studies are initiated. This will allow for correlation of the cell data with actual in vivo effects.

The drugs to be administered in the present invention, or the combination of drugs to be administered, are tested at doses shown to be effective in vitro but also known to be safe in humans. The doses to be administered are routinely chosen by those of skill in the art by using clinical judgment based on results of in vitro pharmacological assays. In the case of bexarotene, however, the drug already has been approved by the FDA for use in humans to treat cancer. It is commonly being administered orally at doses of from 300 to 750 mg/day, doses that are considered both safe and effective in humans with cancer (Physician's Desk Reference 2009). In patients with psoriasis, however, doses used are in the range of 0.5 to 3 mg/kg/day (Smit, A. et al. 2004. J. Am. Acad. Derm. 51:249-256). In the case of NSAIDs such as ibuprofen, usual doses for treatment of symptoms of osteoarthritis are from 600 to 2400 mg/day, not exceeding 2400 mg/day due to the risk of severe side effects (gastrointestinal bleeding). In the case of hydroxychloroquine, usual doses for treatment of arthritis symptoms are in the range of 200 to 400 mg/day. One of skill in the art could determine optimal doses for testing either in vitro or in vivo based on the known safety and efficacy of these drugs in humans. However, it is possible that doses of any of these drugs could be lowered when used in combination due to potential synergistic effects of the drugs in OA. Alternatively, doses chosen can be ones that are equivalent to an effective dose (ED) such as an ED₁₀, an ED₂₅, an ED₅₀, and an ED₇₅ for inhibiting effects in vitro. The drugs to be tested in the present invention are contemplated for oral administration, the most commonly used route of administration for bexarotene as well as NSAIDs and hydroxychloroquine. However, any other route of administration could also be employed that is deemed appropriate by one of skill in the art, including but not limited to administration subcutaneously, intravenously, intramuscularly, or intra-particularly.

Therefore, the present invention is a method of inhibiting synthesis of matrix metalloproteinase (MMP) in synovial tissues comprising contacting synovial tissue with an effective amount of a rexinoid, such as bexarotene, so that levels of MMP in synovial tissue is decreased. The present invention is also a method for inhibiting the degradation of collagen matrix in synovial tissue comprising contacting synovial tissue with an effective amount of a rexinoid, such as bexarotene, so that the degradation of collagen is inhibited. Finally, the present invention is contemplated as a method for treating the symptoms of osteoarthritis in a patient comprising administering to a patient suffering from symptoms of osteoarthritis an effective amount of a rexinoid, such as bexarotene, so that the symptoms of osteoarthritis are reduced. In one embodiment the symptoms of osteoarthritis to be treated are the degradation of collagen in synovial tissue of the joints of the patient. In the context of the present invention, “an effective amount” of bexarotene is that amount of the drug that has been shown to have statistically and/or biologically significant activity either in vitro or in vivo. In other embodiments, bexarotene is given as a combination treatment for OA such as in combination with an NSAID or hydroxychloroquine.

The following non-limiting examples are provided to further illustrate the present invention.

### EXAMPLES

### Example 1

### Cell Culture

Human cells will be propagated at 37° C. in an atmosphere of 5% CO₂ in Dulbecco's modified Eagle's medium (DMEM; Mediatech, Herndon, Va.) containing 10% fetal bovine serum, 100 units/ml penicillin, 100 μl/ml if streptomycin, and 2 mM glutamine. Cells will be washed 3 times with Hank's balanced salt solution and passaged at a 1:10 dilution using 0.25% trypsin. Cells from passages 10-30 will be used.

### Example 2

### Quantitative Real-time Reverse Transcription Polymerase Chain Reaction (RT-PCR)

Cells will be washed twice with cold 1× phosphate-buffered saline (PBS) and total cellular RNA will be harvested using QIASHREDDED spin columns and an RNEASY Mini kit (QIAGEN, Valencia, Calif.) according to manufacturer's instructions. The RT reaction will be performed using 4 μg of purified total RNA and Molney murine leukemia virus reverse transcriptase (Invitrogen, Carlsbad, Calif.) with oligoT+(dT) or random hexamer primers (Applied Biosystems, Foster City, Calif.) for mRNA or heterogeneous nuclear RNA (hnRNA) studies, respectively. The 40 μl reactions will be incubated at 70° C. for 2 minutes, 42° C. for 60 minutes and then 95° C. for 5 minutes.

RT-PCR is performed using a SYBR Green PCR Master Mix kit (Applied Biosystems, Foster City, Calif.) according to manufacturer's instructions. Briefly, 5 μl of the RT product is used in each 50 μl target gene amplification reaction. Because of its greater abundance, 2 μl of RT product is used in each GAPDH amplification reaction. PCR reactions are run with experimental triplicates and machine duplicates or triplicates for each sample. To enable quantitative comparisons between PCR assays, standard curves are generated along with each experiment. Serial long dilutions ranging from 1 ng to 10 fg are made of ATCC plasmids pCMV-sport6 (GAPDH) and pSP6-MMP-1 (MMP-1). Standards of MMPs 2, 3, 13 and 14 are generated.

Both experimental and standard reactions are run using 125 ng each of the appropriate forward and reverse primers. The mRNA primers used include those listed below in Table 1.

TABLE 1
mRNA Primers

| SEQ ID | | |
|---|---|---|
| NO. | Target | Primer Sequence |
| 1 | GAPDH | 5′-CGACAGTCAGCCGCATCTT-3′ |
| | | (sense) |
| 2 | GAPDH | 5′-CCCCATGGTGTCTGAGCG-2′ |
| | | (antisense) |
| 3 | MMP-1 | 5′-AGCTAGCTCAGGATGAGCG-3′ |
| | | (sense) |
| 4 | MMP-1 | 5′-GCCGATGGGCTGGACAG-3′ |
| | | (antisense) |
| 5 | MMP-2 | 5′-TGGCGATGGATACCCCTTT-3′ |
| | | (sense) |
| 6 | MMP-2 | 5′-TTCTCCCAAGGTCCATAGCTCA-3′ |
| | | (antisense) |
| 7 | MMP-3 | 5′-TTCCGCCTGTCTCAAGATGATAT-3′ |
| | | (sense) |
| 8 | MMP-3 | 5′-AAAGGACAAAGCAGGATCACAGT-3′ |
| | | (antisense) |
| 9 | MMP-9 | 5′-CCTGGGCAGATTCCAAACCT-3′ |
| | | (sense) |
| 10 | MMP-9 | 5′-GCAAGTCTTCCGAGTAGTTTTGGAT-3' |
| | | (antisense) |
| 11 | MMP-13 | 5′-CCAGTGGTGGTGATGAAGATG-3′ |
| | | (sense) |
| 12 | MMP-13 | 5′-ACGGCTAGAAAAGACACCAAA-3′ |
| | | (antisense) |

### Example 3

### Collagen Degradation Assay

The collagen degradation assay is performed as previously described (Huntington, J. T. et al. 2004. J. Biol. Chem. 279:33168-33176; Wyatt, C. A. et al. 2005. Cancer Res. 65:11101-11108). Briefly, fibrillar collagen preparations are made from Vitrogen 100 bovine type I collagen (Cohesion Technologies, Palo Alto, Calif.) according to manufacturer's instructions. The collagen solution is diluted to 2 mg/ml and the pH adjusted to 7.3 using sterile 10×PBS and 0.1 N NaOH. Once neutralized, an equivalent volume of DMEM/LH containing cells is added, with a final collagen concentration of 1 mg/ml and 2.5×10⁵ cells per well of a 6-well plate. The following reagents are added to the collagen/cell suspension of specific experimental wells (concentrations given are per ml of collagen): 100 units/ml aproptinin, 3 μg/ml mouse anti-human MMP-1-neutralizing monoclonal antibody (ab-5; Calbiochem, San Diego, Calif.), 3 μg/ml of anti-FLAG monoclonal antibody, as well as varying concentrations of bexarotene. Upon incubation at 37° C. for 60 minutes, the collagen gels, and 1 ml of DMEM/LH is added on top of the cell-containing collagen plug. After 24 hours of incubation in DMEM/LH, IL-1β (1 ng/ml) is added to the medium to induce MMP production and the subsequent collagen degradation. Approximately 24 hours after the addition of the cytokine, the medium is removed from each well and weighed to quantify the extent of collagen degradation.

What is claimed is:

1. A method of inhibiting synthesis of matrix metalloproteinase (MMP) in synovial tissue comprising contacting synovial tissue with an effective amount of a rexinoid drug so that levels of MMP in synovial tissue are decreased.

2. The method of claim 1, wherein said rexinoid drug is bexarotene.

3. A method for inhibiting the degradation of collagen matrix in synovial tissue comprising contacting synovial tissue and cartilage with an effective amount of bexarotene so that the degradation of collagen is inhibited.

4. The method of claim 3, wherein said rexinoid drug is bexarotene.

5. A method for treating the symptoms of osteoarthritis in a patient comprising administering to a patient suffering from symptoms of osteoarthritis an effective amount of a rexinoid drug so that the symptoms of osteoarthritis are reduced.

6. The method of claim 5, wherein said rexinoid drug is bexarotene.

7. The method of claim 5, wherein said symptoms of osteoarthritis include degradation of collagen in synovial tissue of joints of the patient.

8. The method of claim 5, wherein said bexarotene is administered orally.

9. The method of claim 5, wherein said rexinoid drug is administered in combination with an effective amount of a non-steroidal anti-inflammatory drug or hydroxychloroquine.


SEQUENCE LISTING (12 sequences)

SEQ ID NO: 1 - DNA, 19 bp, Artificial Sequence
  Synthetic oligonucleotide
cgacagtcagccgcatctt

SEQ ID NO: 2 - DNA, 18 bp, Artificial Sequence
  Synthetic oligonucleotide
ccccatggtgtctgagcg

SEQ ID NO: 3 - DNA, 19 bp, Artificial Sequence
  Synthetic oligonucleotide
agctagctcaggatgagcg

SEQ ID NO: 4 - DNA, 17 bp, Artificial Sequence
  Synthetic oligonucleotide
gccgatgggctggacag

SEQ ID NO: 5 - DNA, 19 bp, Artificial Sequence
  Synthetic oligonucleotide
tggcgatggataccccttt

SEQ ID NO: 6 - DNA, 22 bp, Artificial Sequence
  Synthetic oligonucleotide
ttctcccaaggtccatagctca

SEQ ID NO: 7 - DNA, 23 bp, Artificial Sequence
  Synthetic oligonucleotide
ttccgcctgtctcaagatgatat

SEQ ID NO: 8 - DNA, 23 bp, Artificial Sequence
  Synthetic oligonucleotide
aaaggacaaagcaggatcacagt

SEQ ID NO: 9 - DNA, 20 bp, Artificial Sequence
  Synthetic oligonucleotide
cctgggcagattccaaacct

SEQ ID NO: 10 - DNA, 25 bp, Artificial Sequence
  Synthetic oligonucleotide
gcaagtcttccgagtagttttggat

SEQ ID NO: 11 - DNA, 21 bp, Artificial Sequence
  Synthetic oligonucleotide
ccagtggtggtgatgaagatg

SEQ ID NO: 12 - DNA, 21 bp, Artificial Sequence
  Synthetic oligonucleotide
acggctagaaaagacaccaaa